(19) United States
(12) Patent Application Publication
(10) Pub. No.: US 2024/0343725 A1
(43) Pub. Date: Oct. 17, 2024

(54) A PROCESS FOR THE PREPARATION OF GRAPIPRANT AND ITS INTERMEDIATES

(71) Applicant: ZENFOLD SUSTAINABLE TECHNOLOGIES PRIVATE LIMITED, Bangalore (IN)

(72) Inventors: Phaneendrasai Karri, Guntur (IN); Srinivasarao Balla, Bengaluru (IN)

(73) Assignee: ZENFOLD SUSTAINABLE TECHNOLOGIES PRIVATE LIMITED, Bangalore (IN)

(21) Appl. No.: 18/294,172

(22) PCT Filed: Jul. 31, 2022
(86) PCT No.: PCT/IN2022/050689

§ 371 Date: Feb. 1, 2024

(30) Foreign Application Priority Data
Aug. 1, 2021 (IN) 202141034603

(51) Int. Cl. C07D 471/04

(52) U.S. Cl. CPC C07D 471/04

(57) ABSTRACT

The present invention provides a process for the preparation of compound of Formula I,

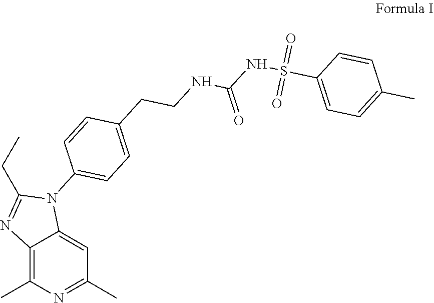

The present invention also provides intermediates formed during the preparation of the compound of Formula I.

### FIELD OF THE INVENTION

The present invention particularly relates to a process for the preparation of Grapiprant and its intermediates.

### BACKGROUND OF THE INVENTION

Drugs from the piprant class are found to be antagonist of the prostaglandin E receptor subtype 4 (EP4), with potential analgesic, immunomodulating and antineoplastic activities. Upon administration of such drug, it selectively binds to and inhibits the binding of prostaglandin E2 (PGE2) and prevents the activation of the EP4 receptor. This inhibits PGE2-EP4 receptor-mediated signaling and prevents proliferation in tumor cells in which the PGE2-EP4 signaling pathway is over-activated. In addition, EP4 receptor inhibition modulates the immune system by preventing both interleukin-23 (IL-23) production and the IL-23-mediated expansion of Th17 cells. As EP4 is expressed by peripheral sensory neurons, blockade of EP4-mediated signaling may induce an analgesic effect.

Particularly, 1-[2-(4-{2-ethyl-4,6-dimethylimidazo[4,5-c]pyridin-1-yl}phenyl)ethyl]-3-(4-ethylphenyl) sulfonylurea is found to be a non-COX inhibiting NSAID that specifically targets the EP4 receptor. It works lower down the inflammatory pathway by blocking some activity of a specific prostaglandin. It has been approved by USFDA to control the pain and inflammation associated with Osteoarthritis in dogs.

Methods for synthesizing compounds such as Grapiprant are known in the prior art documents. For instance, US20210079000 discloses a process for preparation of Grapiprant, the process comprising (a) reacting a compound of Formula (VII) with a compound of Formula (VIII) to obtain a compound of Formula (VI);

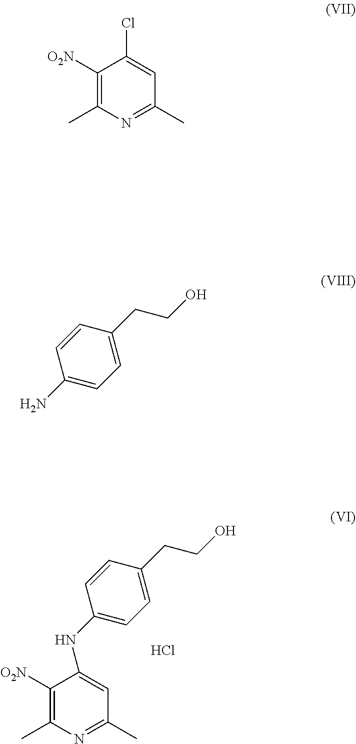

(b) converting the compound of Formula (VI) into a compound of Formula (V);

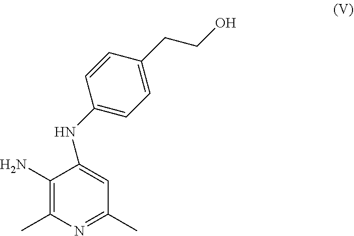 [SMILES: Cc1cc(Nc2ccc(CCO)cc2)c(N)c(C)n1]

(c) converting the compound of Formula (V) into a compound of Formula (IV);

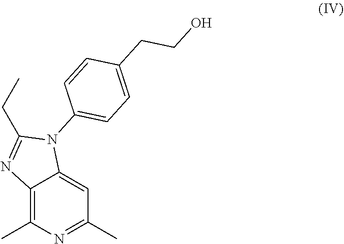 [SMILES: CCc1nc2c(C)nc(C)cc2n1-c1ccc(CCO)cc1]

(d) converting the compound of Formula (IV) into a compound of Formula (III); and

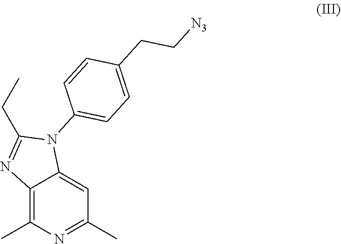 [SMILES: CCc1nc2c(C)nc(C)cc2n1-c1ccc(CCN=[N+]=[N-])cc1]

(e) converting the compound of Formula (III) into a compound of Formula (II).

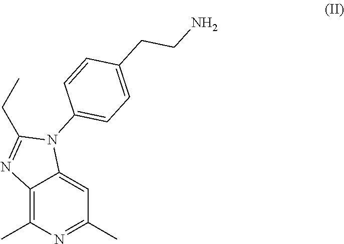 [SMILES: CCc1nc2c(C)nc(C)cc2n1-c1ccc(CCN)cc1]

f) reacting formula II with ethyl(p-tolylsulfonyl) carbamate or p-toluene sulfonyl isocyanate in the presence of a base.

Another prior art WO2020014445 discloses a process for the preparation of Grapiprant, the process comprising:

### Step 1: Preparation of Compound F

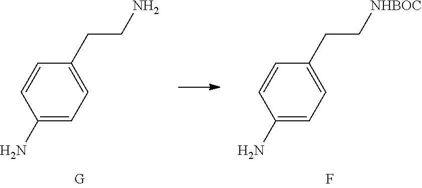

### Step 2. Preparation of Compounds E and D

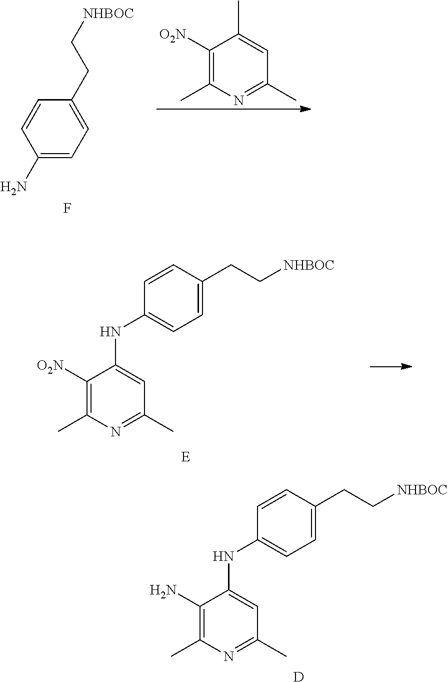

Step 3: Preparation of compounds C. B. and A.

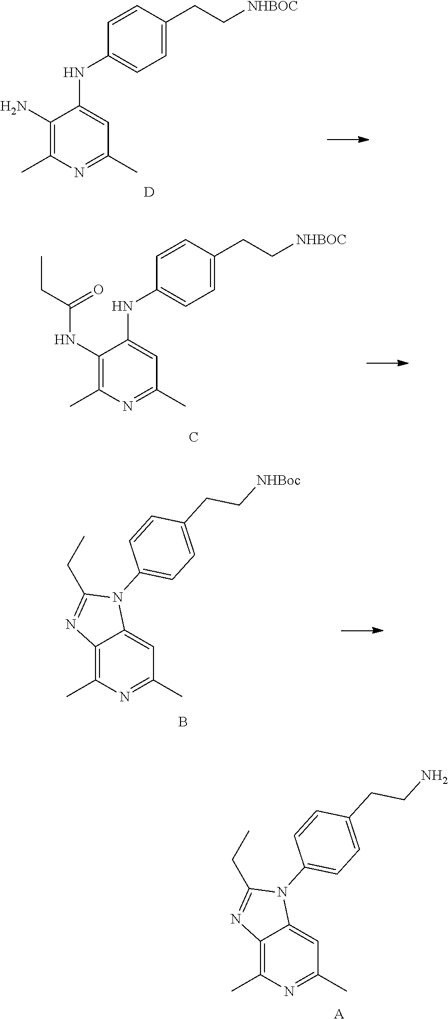

Step 4: Preparation of compound of Formula II

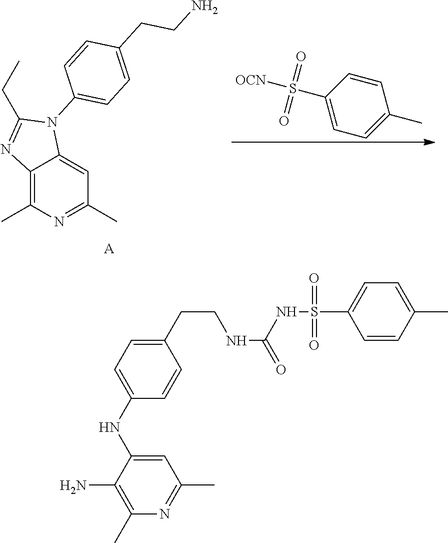

Still another prior art U.S. Pat. No. 6,710,054B2 discloses the preparation of Grapiprant, said process involves the following steps:
(a) coupling reaction of a compound of Formula 1-1 with 4-aminophenethylalcohol wherein X is a leaving group such as halo, mesylate (OMs) or tosylate (OTs) to give a nitroaniline compound of Formula 1-2;
(b) reduction of the resulting nitroaniline compound of Formula 1-2 to give a diamine compound of Formula 1-3;
(c) benzimidazole or imidazopyridine ring formation with the compound of Formula 1-3 to give a compound of Formula 1-4;
(d) hydrolysis of the compound of Formula 1-4 to give a compound of Formula 1-5; conversion of the hydroxy group of the compound 1-5 into a suitable leaving group such as halo, OMs or OTs to give a compound of Formula 1-6;
(e) amination of the compound of Formula 1-6 to give an amino compound of Formula 1-7; and
(f) sulfonylurea formation with the compound of Formula 1-7 to give the compound of Formula (Ia).

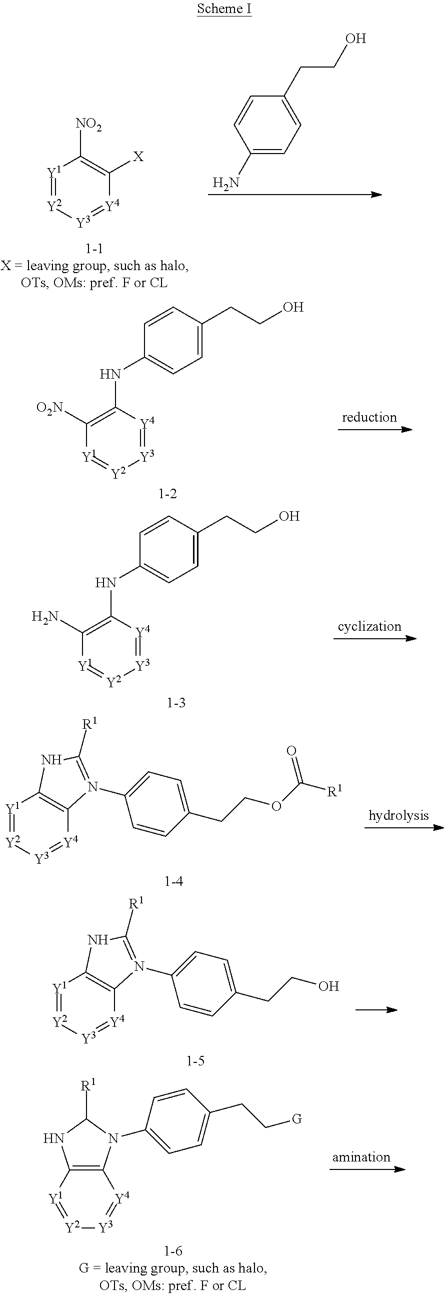

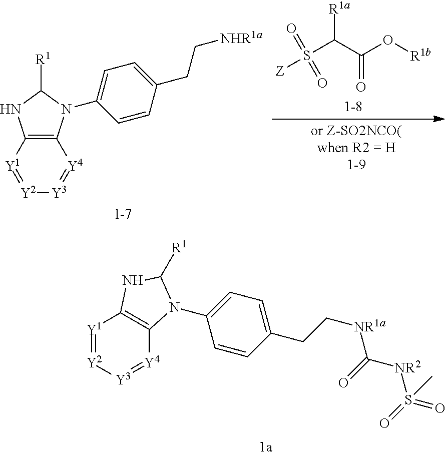

Additionally, U.S. Pat. No. 7,960,407 discloses various crystalline forms of Grapiprant which are designated as Form A and Form B and U.S. Pat. No. 9,265,756 discloses other crystalline form of Grapiprant which are designated as Form X, X₂, X₃, F, K, L, M and N.

The processes disclosed in the prior art patent documents mainly involve the use of potentially hazardous p-toluene sulfonyl isocyanate. Further, the preparation of p-toluene sulfonyl isocyanate requires phosgene which is highly toxic and unsafe. Accordingly, it is envisaged to synthesize Grapiprant and its intermediates without using any hazardous or toxic reactants.

### OBJECTS OF THE INVENTION

An object of the present invention is to provide a novel and high yielding process for the preparation of Grapiprant.

Another object of the present invention is to provide a process for the preparation of highly pure Grapiprant.

Still another object of the present invention is to provide a process for the preparation of Grapiprant which is scalable, environment-friendly and safe.

Still another object of the present invention is to provide a process for the preparation of Grapiprant which obviates the use of isocyanate as a starting material.

### SUMMARY OF THE INVENTION

The present invention provides a process for the preparation of compound of Formula I.

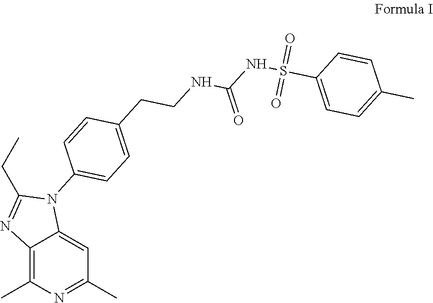

The present invention also provides intermediates of compound of Formula III, IV, V, VII and VIII formed during the preparation of the compound of Formula I.

### DESCRIPTION OF THE INVENTION

Accordingly, in one aspect the present invention provides a process for the preparation of compound of Formula I.

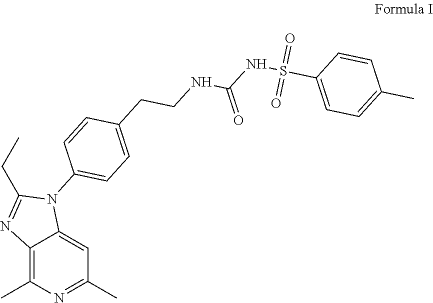

In accordance with the present invention the compound of Formula I is Grapiprant. In one embodiment of the present invention, the process for the preparation of compound of Formula I comprises the steps of:
a) reacting a compound of Formula II

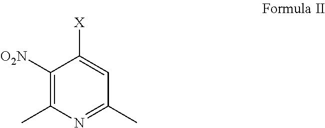

wherein, X is Cl or Br,
with a compound of Formula III

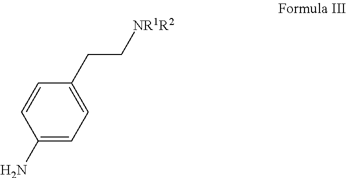

wherein,
R¹ is

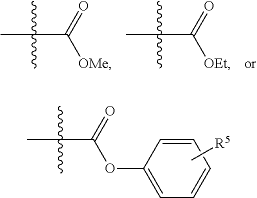

& R⁵ is H, F, Me, OMe, Cl, or nitro; R² is H;
or
R¹ and R² together with the Nitrogen atom to which they are attached is

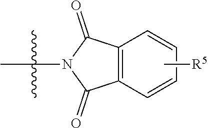

& R⁶ is H, F, Me or nitro,
to obtain a compound of Formula IV;

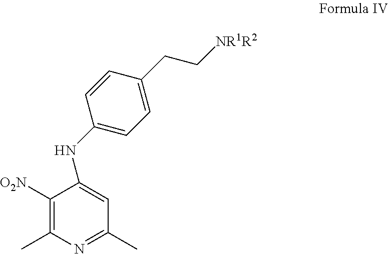

wherein,
R¹ is

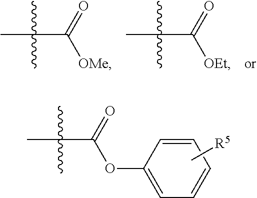

& R⁵ is H, F, Me, OMe, Cl, F or nitro; R² is H;
or
R¹ and R² together with the Nitrogen atom to which they are attached is

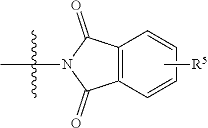

& R⁶ is H, F, Me or nitro,
b) reducing the compound of Formula IV to obtain a compound of Formula V;

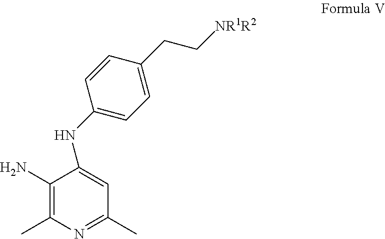

wherein,
R¹ is

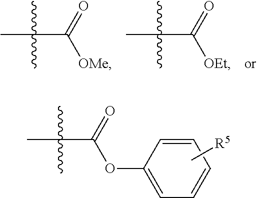

& R⁵ is H, F, Me, OMe, Cl, F or nitro; R² is H;
   or
R¹ and R² together with the Nitrogen atom to which they are attached is

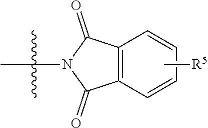

& R⁶ is H, F, Me or nitro,
c) reacting the compound of Formula V with a compound of VI(i) or Formula VI(ii)

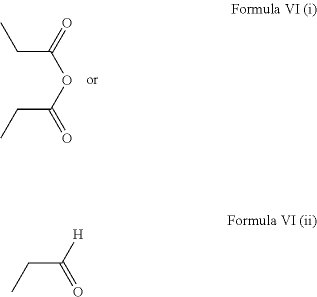

to obtain a compound of Formula VII;

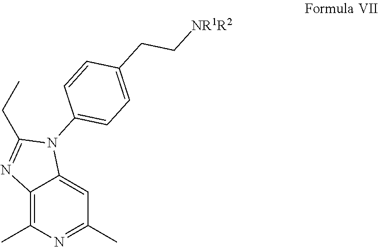

wherein,
R¹ is

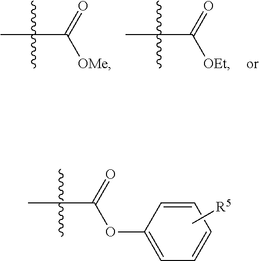

& R⁵ is H, F, Me, OMe, Cl, F or nitro; R² is H;
or
R¹ and R² together with the Nitrogen atom to which they are attached is

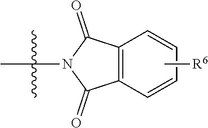

& R⁶ is H, F, Me or nitro,
d) converting the compound of Formula VII into a compound of Formula VIII; and

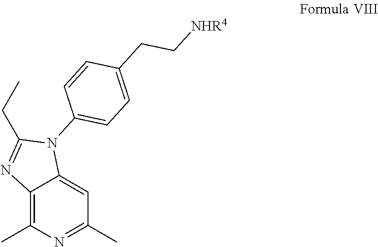

wherein,
R⁴ is H,
e) reacting the compound of Formula VIII

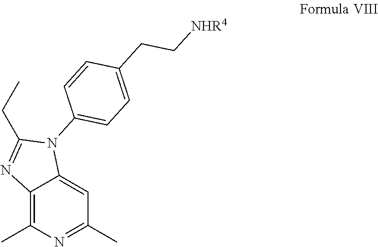

wherein,
R⁴ is H;
with a compound of Formula IXa or IXb

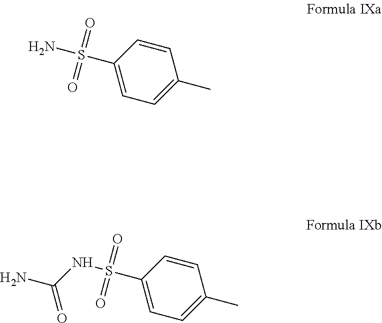

to obtain the compound of formula I.

In one embodiment of the present invention, any or all of the intermediates of compound of Formula III, IV, V, VII and VIII formed during the preparation of the compound of Formula I are isolated.

In another embodiment of the present invention, any or all of the intermediates of compound of Formula III, IV, V, VII and VIII formed during the preparation of the compound of Formula I are not isolated.

In one embodiment of the present invention, the compound of Formula III

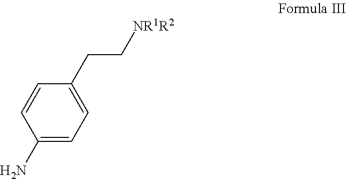

wherein,
   R¹ and R² together with the Nitrogen atom to which they are attached is

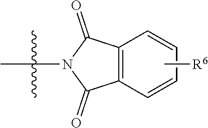

& R⁶ is H, F, Me or nitro
is prepared from 4-nitrobenzaldehyde. In one embodiment, the process involves the following steps:

### Step III-1:

4-Nitrobenzaldehyde is reacted with nitromethane to obtain a compound of Formula III-a.

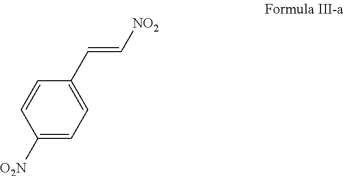 [SMILES: O=[N+]([O-])/C=C/c1ccc([N+](=O)[O-])cc1]

### Step III-2

Reducing the compound of Formula III-a to obtain a compound of Formula III-b.

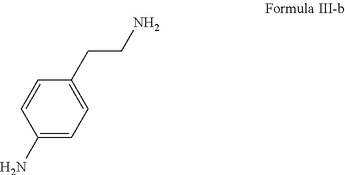

### Step III-3

Reacting the compound of Formula III-b to obtain the compound of Formula III, wherein,
R¹ and R² together with the Nitrogen atom to which they are attached is

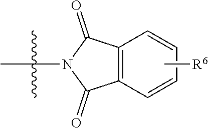

& R⁶ is H, F, Me or nitro.

In another embodiment of the present invention, the process for the preparation of compound of Formula I, comprises the steps of:
a. reacting a compound of Formula X

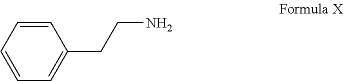

with a compound of Formula XI

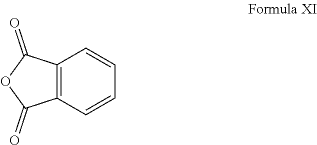 [SMILES: O=C1OC(=O)c2ccccc21]

to obtain a compound of Formula XII,

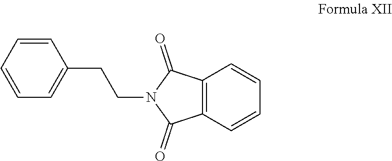 [SMILES: O=C1c2ccccc2C(=O)N1CCc1ccccc1]

b. nitrating the compound of Formula XII to obtain a compound of Formula XIII,

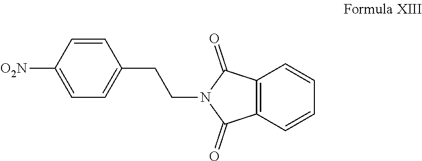 [SMILES: O=C1c2ccccc2C(=O)N1CCc1ccc([N+](=O)[O-])cc1]

c. converting the compound of Formula XIII into a compound of Formula XIV,

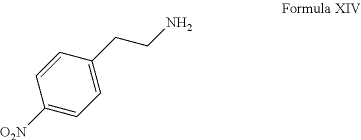 [SMILES: NCCc1ccc([N+](=O)[O-])cc1]

d. reacting the compound of Formula XIV with the compound of Formula IXb

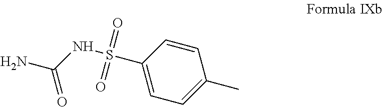

to obtain a compound of Formula XV,

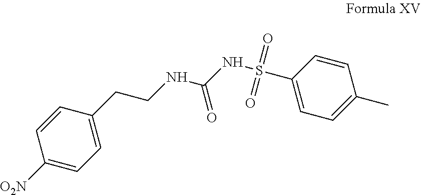 [SMILES: Cc1ccc(S(=O)(=O)NC(=O)NCCc2ccc([N+](=O)[O-])cc2)cc1]

e. reducing the compound of Formula XV to obtain a compound of Formula XVII,

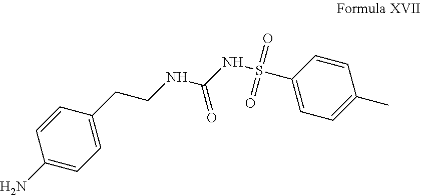 [SMILES: Cc1ccc(S(=O)(=O)NC(=O)NCCc2ccc(N)cc2)cc1]

f. reacting the compound of Formula XVII with the compound of Formula II

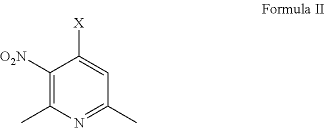

wherein, X is Cl or Br,
to obtain a compound of Formula XVIII,

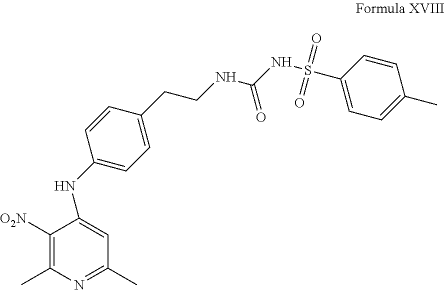

g. reducing the compound of Formula XVIII to obtain a compound of Formula XIX, and

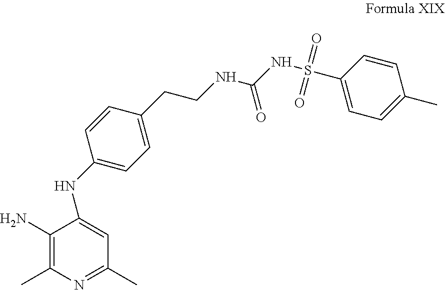 [SMILES: Cc1ccc(S(=O)(=O)NC(=O)NCCc2ccc(Nc3cc(C)nc(C)c3N)cc2)cc1]

h. reacting the compound of Formula XIX with the compound of Formula VI(i) or Formula VI(ii)

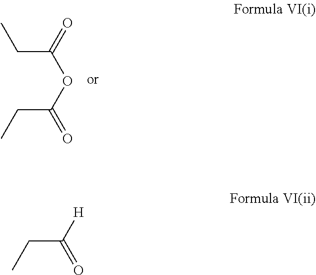

to obtain the compound of Formula I.

Alternatively, the compound of Formula XVII can be obtained in two steps.

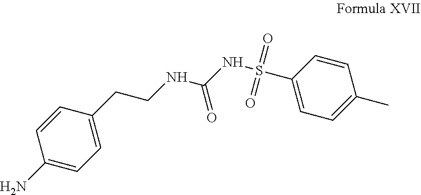 [SMILES: Cc1ccc(S(=O)(=O)NC(=O)NCCc2ccc(N)cc2)cc1]

In the first step, the compound of Formula XIV

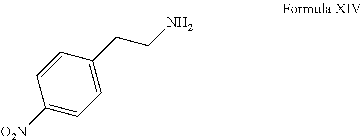 [SMILES: NCCc1ccc([N+](=O)[O-])cc1]

is reduced to obtain a compound of Formula XVI.

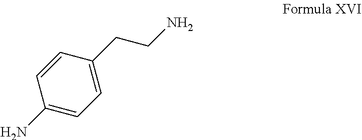

In the second step, the compound of Formula XVI is reacted with the compound of Formula IXb.

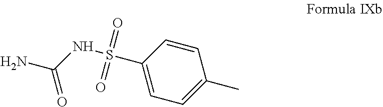

In another aspect, the present invention provides novel intermediates useful for the preparation of the compound of Formula I.

In one embodiment, the present invention provides the compound of Formula IV

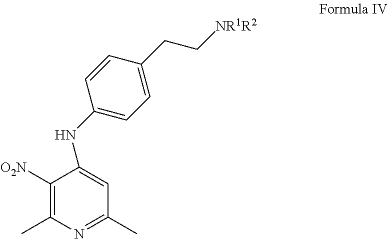

wherein, R¹ is

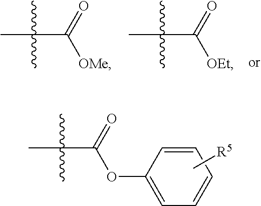

& R⁵ is H, F, Me, OMe, Cl, F or nitro; R² is H;
   or
R¹ and R² together with the Nitrogen atom to which they are attached is

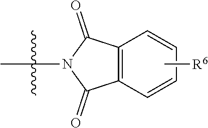

& R⁶ is H, F, Me or nitro.

In another embodiment, the present invention provides the compound of Formula V

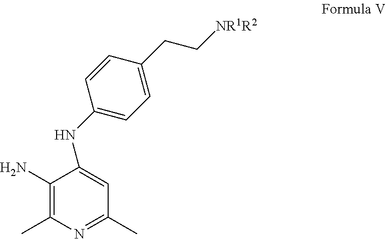

wherein, R¹ is

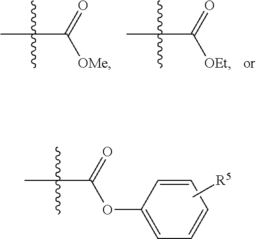

& R⁵ is H, F, Me, OMe, Cl, F or nitro; R² is H;
   or
R¹ and R² together with the Nitrogen atom to which they are attached is

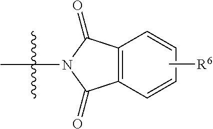

& R⁶ is H, F, Me or nitro.

In still another embodiment, the present invention provides the compound of Formula VII

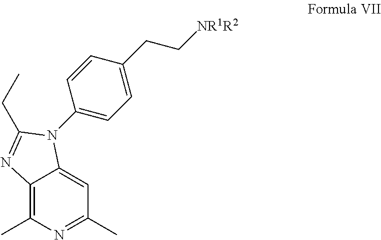

wherein,
R¹ is

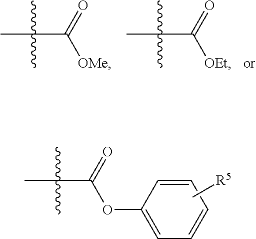

& R⁵ is H, F, Me, OMe, Cl, F or nitro; and R² is H;
   or
R¹ and R² together with the Nitrogen atom to which they are attached is

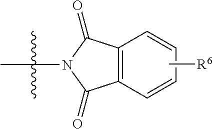

& R⁶ is H, F, Me or nitro.

In still another embodiment, the present invention provides the compound of Formula VIII

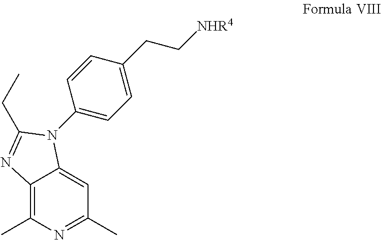

wherein, R⁴ is H.

In still another embodiment, the present invention provides the compound of Formula XVII.

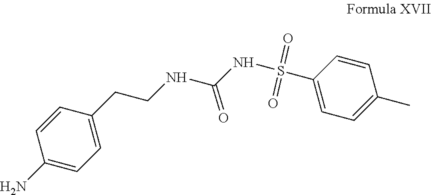 [SMILES: Cc1ccc(S(=O)(=O)NC(=O)NCCc2ccc(N)cc2)cc1]

In still another embodiment, the present invention provides the compound of Formula XVIII.

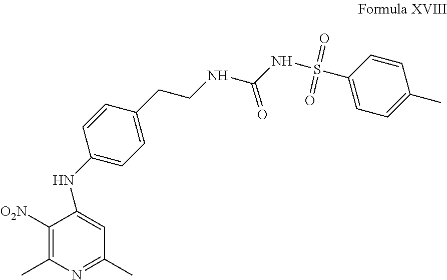

In still another embodiment, the present invention provides the compound of Formula XIX.

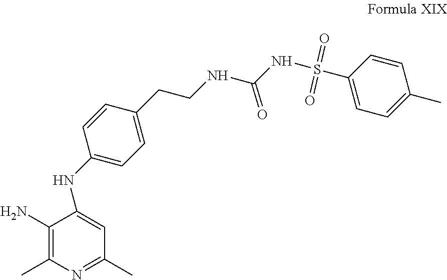 [SMILES: Cc1ccc(S(=O)(=O)NC(=O)NCCc2ccc(Nc3cc(C)nc(C)c3N)cc2)cc1]

The present invention shall now be described with the help of the non-limiting examples. The solvents, reagents, catalyst, temperature pressure conditions, work up mechanism, mode of addition are merely for illustrative purpose. A person skilled in the art can modify, extrapolate or design around the experiments to achieve the results intended by the present invention. Any modifications, extrapolation or any design around shall be the part of the present invention.

### Example 1: Preparation of Grapiprant

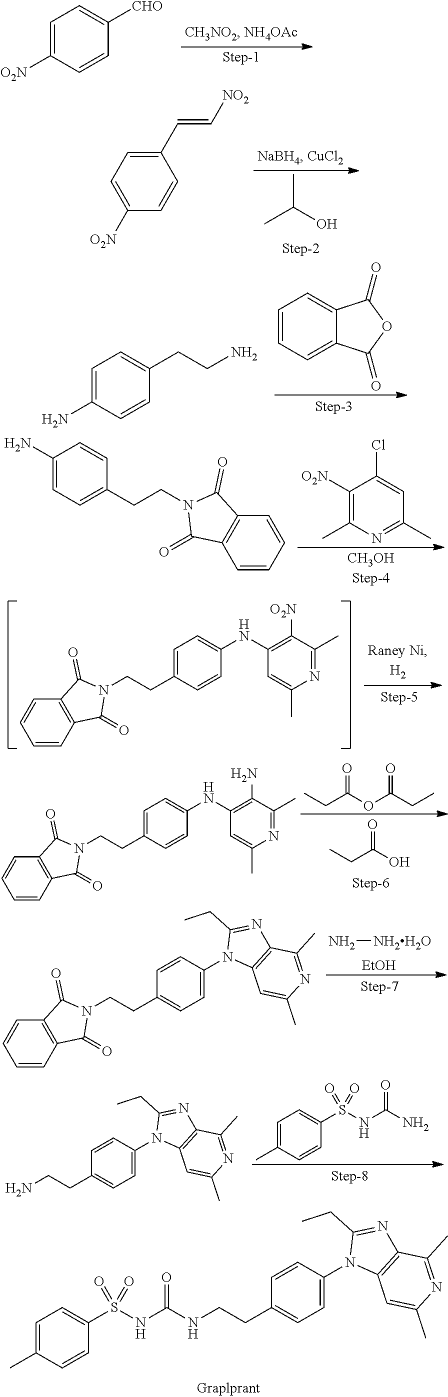

### Step-1:1-nitro-4-(2-nitrovinyl) benzene

A mixture of 4-nitrobenzaldehyde (50 g, 331.1 mmol), nitromethane (50 ml), ammonium acetate (31 g, 500 mmol) in toluene (500 mL) was stirred at 120° C. for 12 hrs. The reaction mass was cooled after completion of the reaction followed by washing the toluene layer by water (3×250 mL) and separating the toluene layer. The toluene layer was dried over Na₂SO₄ and toluene was evaporated under reduced pressure to obtain 1-nitro-4-(2-nitrovinyl) benzene (81 g, 78%).

¹H NMR (300 MHz, CDCl₃): δ 7.79-7.84 (m, 2H), 7.68-7.73 (m, 2H), 7.18-7.31 (m, 5H), 3.92 (t, J=7.8 Hz, 2H), 2.99 (t, J=7.8 Hz, 2H) LC-MS (m/z): 194.05 (M+H)⁺

### Step-2:4-(2-aminoethyl) aniline

A mixture of 1-nitro-4-(2-nitrovinyl) benzene (5 g, 25.9 mmol), sodium borohydride (1 g, 26.3 mmol), copper chloride in isopropyl alcohol (25 ml) was stirred at 25° C. for 10 hours. After completion of the reaction, the reaction mass was quenched in ice, filtered and dried to remove water to obtain 4-(2-aminoethyl) aniline 3.4 g, which was taken to next step without purification.

¹H NMR (300 MHz, CDCl₃): δ 8.14 (d, J=7.8 Hz, 2H), 7.79-7.84 (m, 2H), 7.70-7.84 (m, 2H), 7.41 (d, J=7.8 Hz, 2H), 3.97 (t, J=8.4 Hz, 2H), 3.12 (t, J=8.4 Hz, 2H) LC-MS (m/z): 137.14 (M+H)⁺

### Step-3:2-(4-aminophenethyl) isoindoline-1,3-dione

To a solution of 4-(2-aminoethyl) aniline (5 g, 16.88 mmol) and phthalic anhydride in toluene (50 ml) was stirred at 25° C. for 12 hrs. After completion of the reaction, the reaction mass filtered to obtain 2-(4-aminophenethyl) isoindoline-1,3-dione (4.2 g, 90%).

¹H NMR (300 MHz, CDCl₃): δ 7.79-7.86 (m, 4H), 6.81-(d, J=8.4 Hz, 2H), 6.42-(d, J=8.4 Hz, 2H), 4.85 (s, 2H, NH₂), 3.70 (t, J=7.2 Hz, 2H), 2.72 (t, J=7.2 Hz, 2H) LC-MS (m/z): 267.1 (M+H)⁺

### Step-4:2-(4-((3-amino-2,6-dimethylpyridin-4-yl) amino) phenethyl) iso indoline-1,3-dione

To a solution of 2-(4-aminophenethyl) isoindoline-1,3-dione (2 g, 7.51 mmol) in methanol (20 ml) was added 4-chloro-2,6-dimethyl-3-nitropyridine, (1.40 g, 7.51 mmol) and stirred at 25° C. for 12 hours. After completion of the reaction, Raney Nickel (1 g) was added at 25° C. and stirred under hydrogen for 12 hours. After completion of the reaction, the reaction mass was filter through celite, washed by methanol. Methanol was evaporated to obtain 2-(4-((3-amino-2,6-dimethylpyridin-4-yl) amino) phenethyl) isoindoline-1,3-dione (2.75 g, 94%).

¹H NMR (300 MHz, DMSO-d₆): δ 8.76 (s, 1H), 7.82-7.84 (s, 4H), 7.27 (d, J=8.1 Hz, 2H), 7.15 (d, J=8.1 Hz, 2H), 6.58 (s, 1H), 5.31 (s, 2H), 3.84 (t, J=6.9 Hz, 2H), 2.94 (t, J=6.9 Hz, 2H), 2.38 (s, 3H), 2.30 (s, 3H).

LC-MS (m/z): 387.1 (M+H)⁺

### Step-6:2-(4-(2-ethyl-4,6-dimethyl-1H-imidazo[4,5-c]pyridin-1-yl) phenethyl) isoindoline-1,3-dione

To a solution of 2-(4-((3-amino-2,6-dimethylpyridin-4-yl) amino) phenethyl) isoindoline-1,3-dione (2.75 g, 7.12 mmol) in tetrahydrofuran (30 ml) was added triethyl amine, (2.16 g, 21.35 mmol) at 0° C. followed by propionic anhydride diluted in tetrahydrofuran (1.85 g, 14.23 mmol) at 0° C. and stirred at 25° C. 12 hours then heated to reflux. After completion of the reaction, the reaction mass was diluted by dichloromethane and washed with water. A dichloromethane layer was separated, dried over anhydrous sodium sulphate and evaporated to obtain 2-(4-(2-ethyl-4,6-dimethyl-1H-imidazo[4,5-c]pyridin-1-yl) phenethyl) isoindoline-1,3-dione (2.78 g, 92%).

¹H NMR (300 MHz, CDCl₃): δ 7.82-7.84 (m, 2H), 7.70-7.73 (m, 2H), 7.44 (d, J=8.4 Hz, 2H), 7.21 (d, J=8.4 Hz, 2H), 6.66 (s, 1H), 4.01 (t, J=8.1 Hz, 2H), 3.11 (t, J=8.1 Hz, 2H), 2.86 (s, 3H), 2.77 (q, J=7.5 Hz, 2H), 2.54 (s, 3H), 1.24 (t, J=7.5 Hz, 2H).

LC-MS (m/z): 425.1 (M+H)⁺

### Step-7:2-(4-(2-ethyl-4,6-dimethyl-1H-imidazo[4,5-c]pyridin-1-yl) phenyl) ethan-1-amine

To a solution of 2-(4-(2-ethyl-4,6-dimethyl-1H-imidazo[4,5-c]pyridin-1-yl) phenethyl) isoindoline-1,3-dione (2.78 g, 6.55 mmol) in isopropyl alcohol (30 ml) was added hydrazine monohydrate (99%) (1.31 g, 26.20 mmol) at 25° C. and stirred at reflux for 2 hours. After completion of the reaction, the reaction mass was filter to remove phthalyl hydrazide and evaporated to remove isopropyl alcohol under reduced pressure to afford crude compound which was further diluted by water and extracted by dichloromethane to obtain pure 2-(4-(2-ethyl-4,6-dimethyl-1H-imidazo[4,5-c]pyridin-1-yl) phenyl) ethan-1-amine (1.8 g, 93%).

¹H NMR (300 MHz, CDCl₃): δ 7.41 (d, J=8.1 Hz, 2H), 7.25 (d, J=8.1 Hz, 2H), 6.72 (s, 1H), 3.06 (t, J=6.9 Hz, 2H), 2.85-2.2.89 (m, 5H), 2.82, (q, J=7.5, 2H), 2.52 (s, 3H), 1.29 (t, J=7.5 Hz, 2H).

LC-MS (m/z): 295.2 (M+H)⁺

### Step-8: N-((4-(2-ethyl-4,6-dimethyl-1H-imidazo[4,5-c]pyridin-1-yl) phenethyl) carbamoyl)-4-methylbenzenesulfonamide (Grapiprant)

To a solution of 2-(4-(2-ethyl-4,6-dimethyl-1H-imidazo[4,5-c]pyridin-1-yl) phenyl) ethan-1-amine (1.8 g, 6.11 mmol) in toluene (5 ml) was added N-carbamoyl-4-methylbenzene-1-sulfonamide (the compound of Formula IXb), (1.31 g, 6.11 mmol) at 25° C. and refluxed for 1 hour. After completion of the reaction, the reaction mass was cooled to afford the crude compound which was further purified by crystallization form acetone to obtain N-((4-(2-ethyl-4,6-dimethyl-1H-imidazo[4,5-c]pyridin-1-yl) phenethyl) carbamoyl)-4-methylbenzenesulfonamide (1.5 g, 51%).

¹H NMR (300 MHZ, CDCl₃): δ 7.75 (d, J=8.4 Hz, 2H), 7.38 (d, J=8.4 Hz, 2H), 7.26 (d, J=7.8 Hz, 2H), 7.21 (d, J=7.8 Hz, 2H), 6.77 (s, 2H), 3.53 (q, J=6.9 Hz, 2H), 2.89-2.2.94 (m, 5H), 2.79, (q, J=7.5, 2H), 2.53 (s, 3H), 2.38 (s, 3H), 1.28 (t, J=7.5 Hz, 2H).

LC-MS (m/z): 492.1 (M+H)⁺

### Example 2: Preparation of Grapiprant

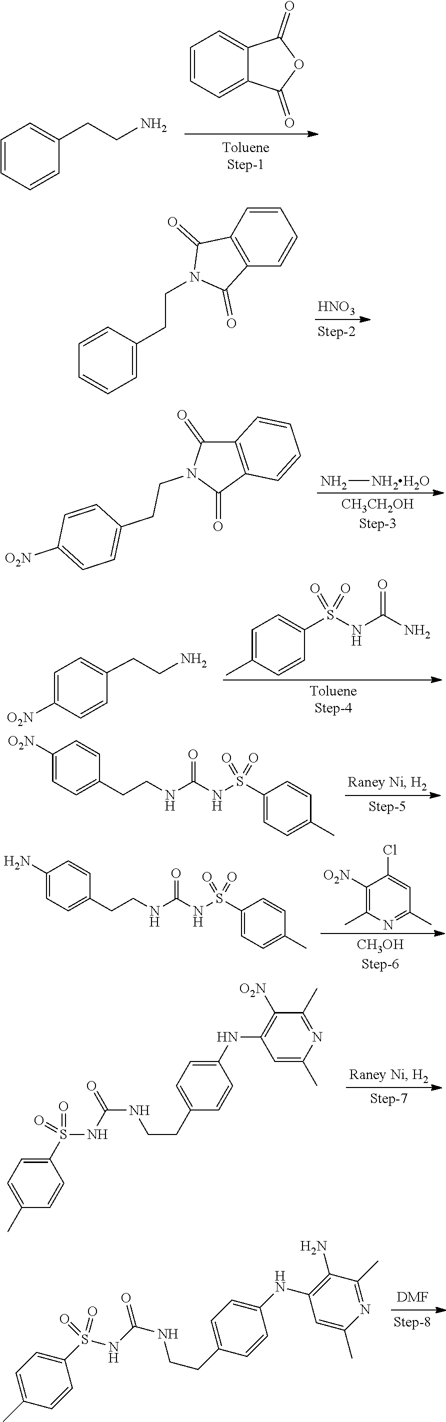

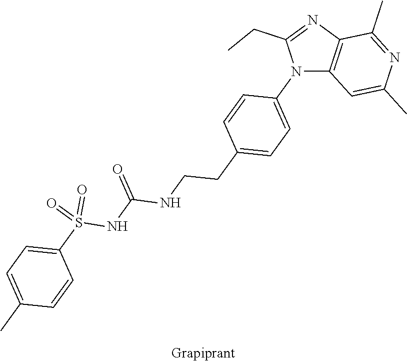

### Step-1:2-phenethylisoindoline-1,3-dione

To a solution of 2-phenylethan-1-amine (50 g, 412.6 mmol) in toluene (500 mL) was added phthalic anhydride (61.1 g, 412.60 mmol) at 25° C. and stirred at reflux 12 hours. After completion of the reaction, the reaction mass was cooled, the toluene layer was washed with water (3×250 mL) and separated. The separated toluene layer was dried over anhydrous sodium sulphate and toluene was evaporated under reduced pressure to obtain 2-phenethylisoindoline-1,3-dione (81 g, 78%).

¹H NMR (300 MHz, CDCl₃): δ 7.79-7.84 (m, 2H), 7.68-7.73 (m, 2H), 7.18-7.31 (m, 5H), 3.92 (t, J=7.8 Hz, 2H), 2.99 (t, J=7.8 Hz, 2H)

LC-MS (m/z): 252.05 (M+H)⁺

### Step-2:2-(4-nitrophenethyl) isoindoline-1,3-dione

2-phenethylisoindoline-1,3-dione (20 g, 79.59 mmol) was added to fuming nitric acid (1 vol.) lot wise below 10° C. and stirred at the same temperature for 45 minutes. After completion of the reaction, the reaction mass was quenched in ice, filtered to remove all water to afford the crude compound 23 g (60:40 ratio), which was further crystalized by isopropyl alcohol (10 vol.) by reflux for 12 h and filtered hot to obtain pure 2-(4-nitrophenethyl) isoindoline-1,3-dione (10 g, 42%).

¹H NMR (300 MHZ, CDCl₃): δ 8.14 (d, J=7.8 Hz, 2H), 7.79-7.84 (m, 2H), 7.70-7.84 (m, 2H), 7.41 (d, J=7.8 Hz, 2H), 3.97 (t, J=8.4 Hz, 2H), 3.12 (t, J=8.4 Hz, 2H)

LC-MS (m/z): 297.14 (M+H)⁺

### Step-3:2-(4-nitrophenyl) ethan-1-amine

To a solution of 2-(4-nitrophenethyl) isoindoline-1,3-dione (24 g, 81 mmol) in methanol (360 mL, 15 vol) was added hydrazine monohydrate, 99% (16.22 mL, 324.02 mmol) at 25° C. and stirred at reflux for 3 hours. After completion of the reaction, the reaction mass was cooled to 25° C., filtered and evaporated to remove methanol. The reaction mass was diluted by ethyl acetate, washed with water (3×100 mL), separated the ethyl acetate layer, dried over anhydrous sodium sulphate and evaporated to remove ethyl acetate under reduced pressure to obtain 2-(4-nitrophenyl) ethan-1-amine (12 g, 89%).

¹H NMR (300 MHz, CDCl₃): δ 8.16 (d, J=8.7 Hz, 2H), 7.55 (d, J=8.7 Hz, 2H), 3.02 (t, J=6.9 Hz, 2H), 2.85 (t, J=6.9 Hz, 2H)

LC-MS (m/z): 166.94 (M+H)⁺

### Step-4:4-methyl-N-((4-nitrophenethyl) carbamoyl) benzene sulfonamide

To a solution of 2-(4-nitrophenyl) ethan-1-amine (3.3 g, 19.86 mmol) in toluene (50 mL) was added N-carbamoyl-4-methylbenzenesulfonamide, (4.25 g, 19.86 mmol) at 25° C. and stirred at reflux for 6 hours. After completion of the reaction, the reaction mass was cooled to 25° C., filtered, and washed by hexane to remove traces of toluene to obtain 4-methyl-N-((4-nitrophenethyl) carbamoyl) benzene-sulfonamide (6.7 g, 92%).

¹H NMR (300 MHz, CDCl₃): δ 8.12 (d, J=8.7 Hz, 2H), 7.61 (d, J=8.4 Hz, 2H), 7.28-7.33 (m, 4H), 6.64 (t, J=8.4 Hz, 2H), 3.53 (q, J=6.9 Hz, 2H), 2.93 (t, J=6.9 Hz, 2H)

LC-MS (m/z): 363.9 (M+H)⁺

### Step-5: N-((4-aminophenethyl) carbamoyl)-4-methylbenzenesulfonamide

To a solution of 4-methyl-N-((4-nitrophenethyl) carbamoyl) benzene sulfonamide (1.2 g, 3.30 mmol) in methanol (60 mL) was added Raney Nickel, (500 mg) at 25° C. and stirred under hydrogen balloon for 3 days. After completion of the reaction, the reaction mass was filtered through celite, washed by methanol, evaporated to obtain N-((4-aminophenethyl) carbamoyl)-4-methylbenzenesulfonamid (850 mg, 77%).

¹H NMR (300 MHz, CDCl₃): δ 7.59 (d, J=8.4 Hz, 2H), 7.27 (d, J=8.1 Hz, 2H), 6.96 (d, J=8.4 Hz, 2H), 6.65 (d, J=8.4 Hz, 2H), 6.53 (s, 1H), 3.42 (q, J=6.9 Hz, 2H), 2.69 (t, J=6.9 Hz, 2H), 2.43 (s, 3H).

LC-MS (m/z): 334.11 (M+H)⁺

### Step 5 (a): N-((4-aminophenethyl) carbamoyl)-4-methylbenzenesulfonamide

To a solution of 4-(2-aminoethyl) aniline (3.3 g, 24.26 mmol) in toluene (50 mL) was added N-carbamoyl-4-methylbenzenesulfonamide, (4.25 g, 19.86 mmol) at room temperature, allowed to stir reaction at 50° C. for 6 hrs. After completion of reaction, the resultant mass was cooled to room temperature and filtered the precipitate compound and the filter cake was washed by hexane to remove traces of toluene, to afford the titled compound (6.3 g, 78%) as pale-yellow solid.

### Step-6: N-((4-((2,6-dimethyl-3-nitropyridin-4-yl) amino) phenethyl) carbamoyl)-4-methylbenzenesulfonamide

To a solution of N-((4-aminophenethyl) carbamoyl)-4-methyl benzene sulfonamide (2.0 g, 6.0 mmol) in methanol (10 mL) was added 4-chloro-2,6-dimethyl-3-nitropyridine (1.12 g, 6.0 mmol) at 25° C. and stirred at 50° C. for 24 hours. After completion of the reaction, the reaction mass was cooled and evaporated to remove methanol to obtain-((4-((2,6-dimethyl-3-nitropyridin-4-yl) amino) phenethyl) carbamoyl)-4-methylbenzenesulfonamide (2.5 g, 5.1 mmol, 85%).

¹H NMR (300 MHz, CDCl₃): δ 8.74 (s, 1H), 7.66 (d, J=8.4 Hz, 2H), 7.24-7.31 (m, 4H), 7.19 (d, J=8.4 Hz, 2H), 6.66 (s, 2H), 3.52 (q, J=7.2 Hz, 2H), 2.86 (t, J=6.9 Hz, 2H), 2.73 (s, 3H), 2.43 (s, 3H), 2.33 (s, 3H).

LC-MS (m/z): 485.1 (M+H)⁺

### Step-7: N-((4-((3-amino-2,6-dimethylpyridin-4-yl) amino) phenethyl) carbamoyl)-4-methylbenzenesulfonamide

To a solution of N-((4-((2,6-dimethyl-3-nitropyridin-4-yl) amino) phenethyl) carbamoyl)-4-methylbenzene sulfonamide (2.0 g, 4.12 mmol) in methanol (10 mL) was added Raney Nickel, (100 mg at 25° C. and stirred under hydrogen balloon (˜1 Kg pressure) for 24 h. After completion of the reaction, the reaction mass was filtered through celite, washed by methanol and evaporated to remove the solvent to obtain N-((4-((3-amino-2,6-dimethylpyridin-4-yl) amino) phenethyl) carbamoyl)-4-methylbenzenesulfonamide (1.65 g, 88%).

LC-MS (m/z): 454.2 (M+H)⁺.

### Step-8: N-((4-(2-ethyl-4,6-dimethyl-1H-imidazo[4,5-c]pyridin-1-yl) phenethyl) carbamoyl)-4-methylbenzenesulfonamide (Grapiprant)

To a solution of N-((4-((3-amino-2,6-dimethylpyridin-4-yl) amino) phenethyl) carbamoyl)-4-methylbenzenesulfonamide (1.0 g, 2.2 mmol) in dimethyl formamide (5 mL) was added propionic anhydride, (343 mg, 2.64 mmol) and triethylamine (330 mg, 3.3 mmol) at 25° C. stirred the reaction at reflux for 1 h. After completion of the reaction, the reaction mass was cooled and dimethylformamide was removed under reduced pressure to afford the crude compound, which was dissolved in dichloromethane (10 mL) and washed with 10% aqueous sodium bicarbonate solution. Dichloromethane was distill-off to get tech-grade product, which was recrystallized form acetone to obtain N-((4-(2-ethyl-4,6-dimethyl-1H-imidazo[4,5-c]pyridin-1-yl) phenethyl) carbamoyl)-4-methylbenzenesulfonamide (0.876 g, 1.78 mmol, 81%).

¹H NMR (300 MHz, DMSO-d₆): δ 7.75 (d, J=8.4 Hz, 2H), 7.38 (d, J=8.4 Hz, 2H), 7.26 (d, J=7.8 Hz, 2H), 7.21 (d, J=7.8 Hz, 2H), 6.77 (s, 2H), 3.53 (q, J=6.9 Hz, 2H), 2.89-2.2.94 (m, 5H), 2.79, (q, J=7.5, 2H), 2.53 (s, 3H), 2.38 (s, 3H), 1.28 (t, J=7.5 Hz, 2H).

LC-MS (m/z): 492.1 (M+H)⁺

### Step-8: N-((4-(2-ethyl-4,6-dimethyl-1H-imidazo[4,5-c]pyridin-1-yl) phenethyl) carbamoyl)-4-methylbenzenesulfonamide (Grapiprant)

Alternative process: To a solution of N-((4-((3-amino-2,6-dimethylpyridin-4-yl) amino) phenethyl) carbamoyl)-4-methylbenzenesulfonamide (1.0 g, 2.2 mmol) in dimethylformamide (5 mL) was added propionaldehyde, (255 mg, 4.4 mmol) and 2,3-Dichloro-5,6-dicyano-1,4-benzoquinone (499 mg, 2.2 mmol) at 25° C. stirred the reaction at reflux for 1 h. After completion of the reaction, the reaction mass was cooled and dimethylformamide was removed under reduced pressure to afford the crude compound, which was dissolved in dichloromethane (10 mL) and washed with 10% aqueous sodium bicarbonate solution. Dichloromethane was distill-off to get tech-grade product, which was recrystallized form acetone to obtain N-((4-(2-ethyl-4,6-dimethyl-1H-imidazo[4,5-c]pyridin-1-yl) phenethyl) carbamoyl)-4-methylbenzenesulfonamide (0.876 g, 1.78 mmol, 81%).

LC-MS (m/z): 492.1 (M+H)⁺

1H NMR (300 MHz, CDCl₃): δ 7.81-7.79 (d, 2H), 7.41-7.39 (d, 3H), 7.28-7.20 (m, 4H), 6.91 (s, 1H), 6.83 (s, 1H), 3.54-3.52 (d, 2H), 2.94-2.90 (m, 4H), 2.83-2.77 (q, 2H), 2.77 (s, 3H), 2.56 (s, 3H), 1.31-1.27 (t, 3H).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A process for the preparation of compound of Formula I, said process comprises the following steps: 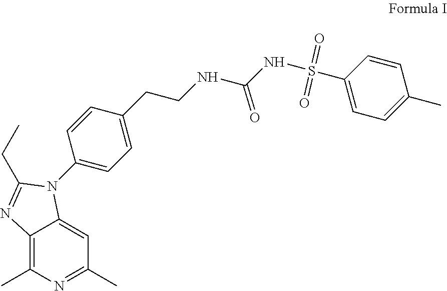
a. reacting a compound of Formula II 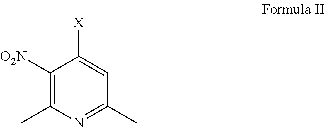
wherein, X is Cl or Br,
with a compound of Formula III 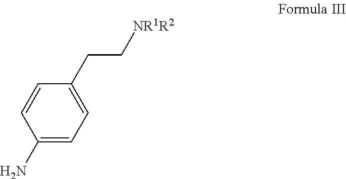
wherein,
R¹ is 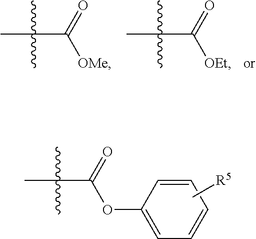
& R⁵ is H, F, Me, OMe, Cl, or nitro; R² is H;
or
R¹ and R² together with the Nitrogen atom to which they are attached is 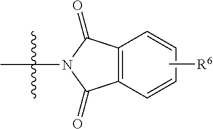
& R⁶ is H, F, Me or nitro,
to obtain a compound of Formula IV; 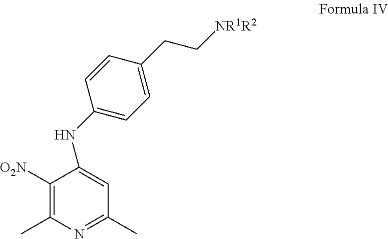
wherein,
R¹ is 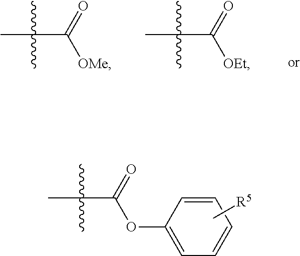
& R⁵ is H, F, Me, OMe, Cl, F or nitro; R² is H;
or
R¹ and R² together with the Nitrogen atom to which they are attached is 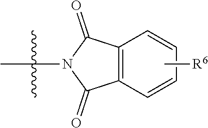
& R⁶ is H, F, Me or nitro,
b. reducing the compound of Formula IV to obtain a compound of Formula V; 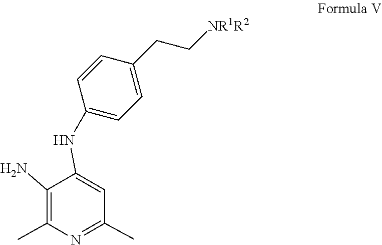
wherein,
R¹ is 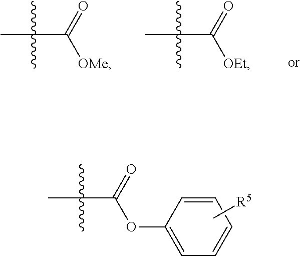
& R⁵ is H, F, Me, OMe, Cl, F or nitro; R² is H;
or
R¹ and R² together with the Nitrogen atom to which they are attached is 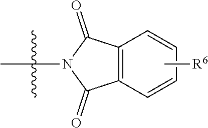
& R⁶ is H, F, Me or nitro,
c. reacting the compound of Formula V with a compound of Formula VI(i) or Formula VI(ii), 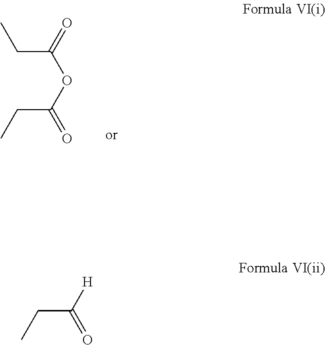
to obtain a compound of Formula VII; 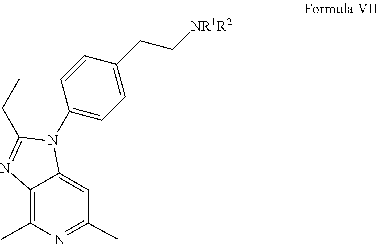
wherein,
R¹ is 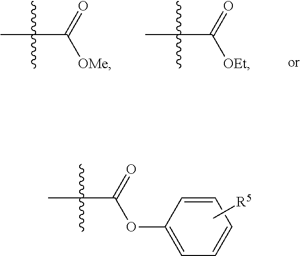
& R⁵ is H, F, Me, OMe, Cl, F or nitro; R² is H;
or
R¹ and R² together with the Nitrogen atom to which they are attached is 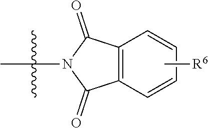
& R⁶ is H, F, Me or nitro,
d. converting the compound of Formula VII into a compound of Formula VIII; and 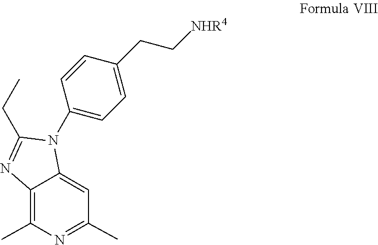
wherein,
R⁴ is H,
e. reacting the compound of Formula VIII 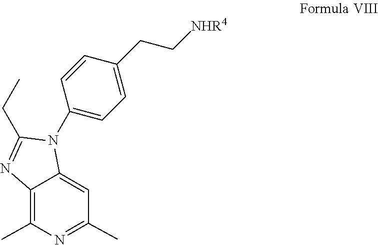
wherein,
R⁴ is H;
with a compound of Formula IXa or IXb 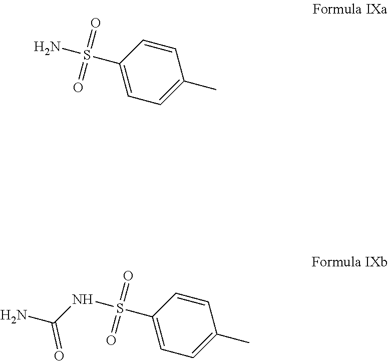
to obtain the compound of Formula I.

2. The process as claimed in claim 1, wherein the compound of Formula III is 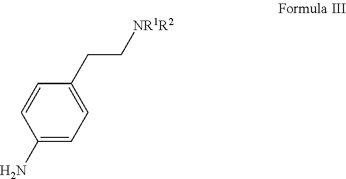
wherein,
R¹ and R² together with the Nitrogen atom to which they are attached is 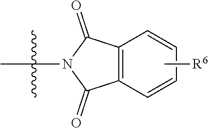
& R⁶ is H, F, Me or nitro

3. The process as claimed in claim 2, wherein the compound of Formula III is prepared from 4-nitrobenzaldehyde, said process comprising the following steps:
i) reacting 4-Nitrobenzaldehyde with nitromethane to obtain a compound of Formula III-a, 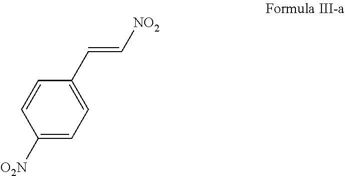 [SMILES: O=[N+]([O-])/C=C/c1ccc([N+](=O)[O-])cc1]
ii) reducing the compound of Formula III-a to obtain a compound of Formula III-b, and 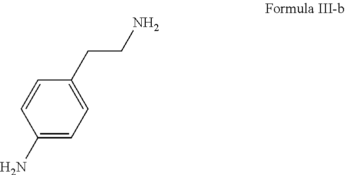
iii) converting the compound of Formula III-b into the compound of Formula III.

4. A process for the preparation of compound of Formula I comprises the following steps:
i. reacting a compound of Formula X 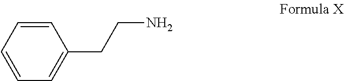
with a compound of Formula XI 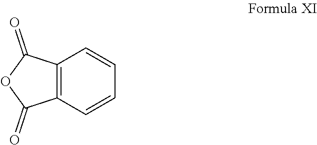 [SMILES: O=C1OC(=O)c2ccccc21]
to obtain a compound of Formula XII, 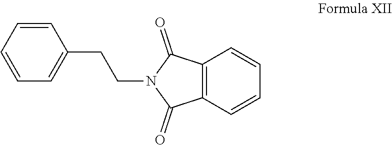 [SMILES: O=C1c2ccccc2C(=O)N1CCc1ccccc1]
ii. nitrating the compound of Formula XII to obtain a compound of Formula XIII, 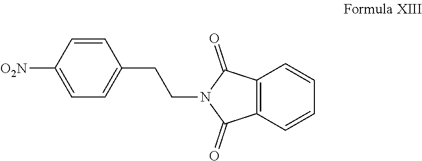 [SMILES: O=C1c2ccccc2C(=O)N1CCc1ccc([N+](=O)[O-])cc1]
iii. converting the compound of Formula XIII into a compound of Formula XIV, 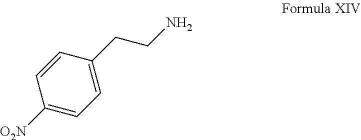 [SMILES: NCCc1ccc([N+](=O)[O-])cc1]
iv. reacting the compound of Formula XIV with the compound of Formula IXb 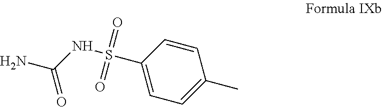
to obtain a compound of Formula XV, 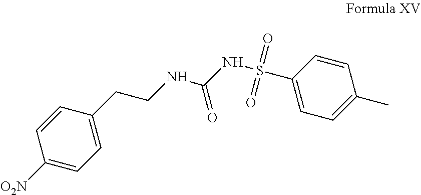 [SMILES: Cc1ccc(S(=O)(=O)NC(=O)NCCc2ccc([N+](=O)[O-])cc2)cc1]
v. reducing the compound of Formula XV to obtain a compound of Formula XVII, 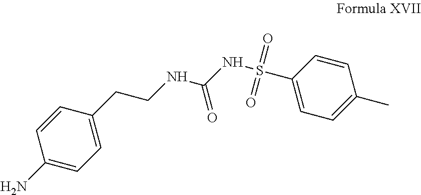 [SMILES: Cc1ccc(S(=O)(=O)NC(=O)NCCc2ccc(N)cc2)cc1]
vi. reacting the compound of Formula XVII with the compound of Formula II 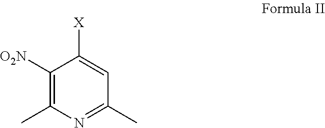
wherein, X is Cl or Br,
to obtain a compound of Formula XVII, 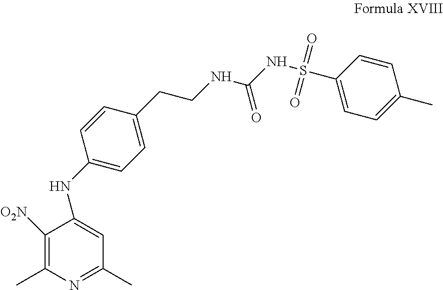
vii. reducing the compound of Formula XVIII to obtain a compound of Formula XIX, and 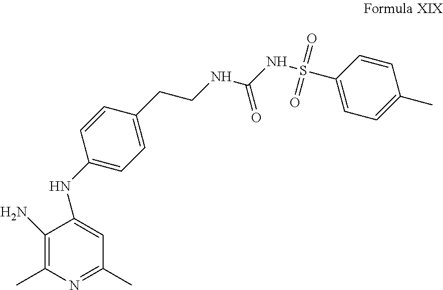
viii. reacting the compound of Formula XIX with the compound of Formula VI(i) or Formula VI(ii), 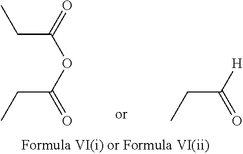
to obtain the compound of Formula I.

5. The process as claimed in claim 4, wherein the compound of Formula XVII 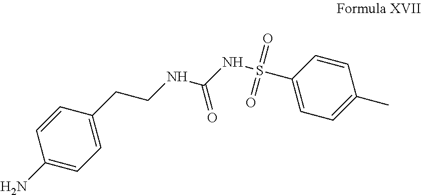 [SMILES: Cc1ccc(S(=O)(=O)NC(=O)NCCc2ccc(N)cc2)cc1]
is obtained by a process comprising reducing a compound of Formula XIV 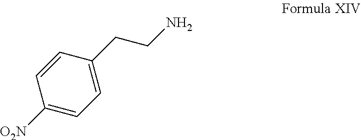 [SMILES: NCCc1ccc([N+](=O)[O-])cc1]
to obtain a compound of Formula XVI, and 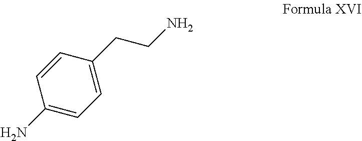
reacting the compound of Formula XVI with the compound of Formula IXb.

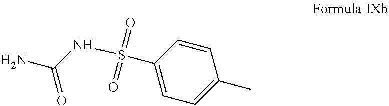

6. A compound of Formula IV 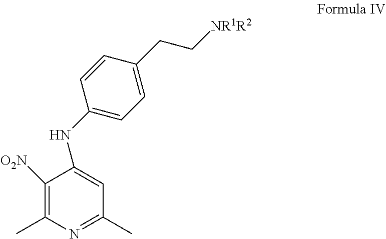
wherein, R¹ is 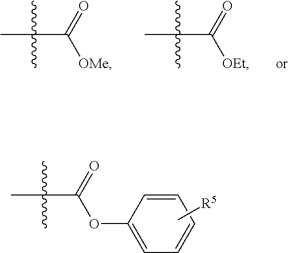
& R⁵ is H, F, Me, OMe, Cl, F or nitro; R² is H;
or
R¹ and R² together with the Nitrogen atom to which they are attached is 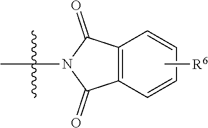
& R⁶ is H, F, Me or nitro.

7. A compound of Formula V 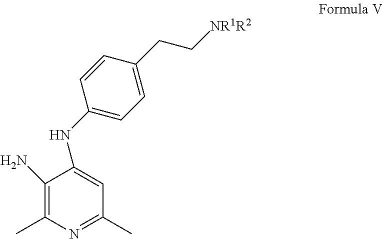
wherein, R¹ is 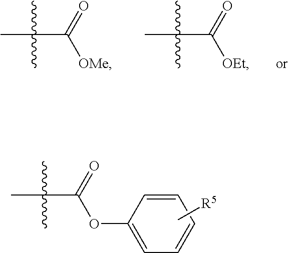
& R⁵ is H, F, Me, OMe, Cl, F or nitro; R² is H;
or
R¹ and R² together with the Nitrogen atom to which they are attached is 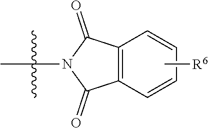
& R⁶ is H, F, Me or nitro.

8. A compound of Formula VII 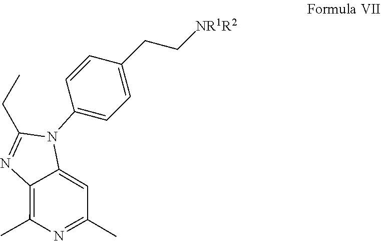
wherein,
R¹ is 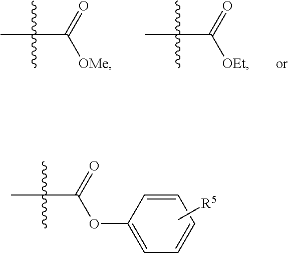
& R⁵ is H, F, Me, OMe, Cl, F or nitro; and R² is H;
or
R¹ and R² together with the Nitrogen atom to which they are attached is 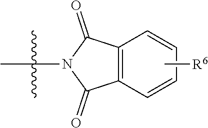
& R⁶ is H, F, Me or nitro.

9. A compound of Formula VIII 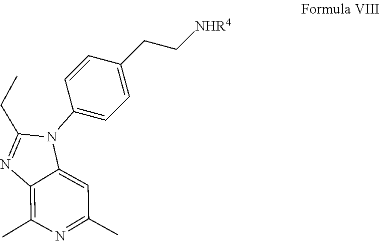
wherein, R⁴ is H.

10. A compound of Formula XVII

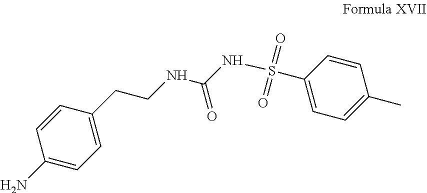 [SMILES: Cc1ccc(S(=O)(=O)NC(=O)NCCc2ccc(N)cc2)cc1]

11. A compound of Formula XVIII

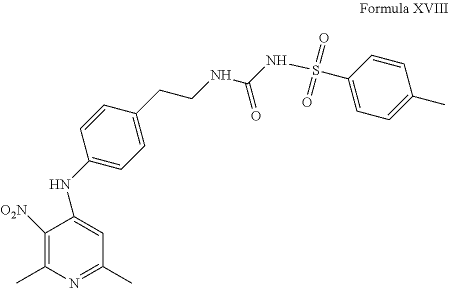

12. A compound of Formula XIX [chemical structure]
